(19) United States
(12) Patent Application Publication
(10) Pub. No.: US 2006/0142637 A1
(43) Pub. Date: Jun. 29, 2006

(54) Surgical instrument for the treatment of female urinary incontinence and methods of use

(76) Inventor: Atamjit Gill, St. John's (CA)

Correspondence Address: CHRISTENSEN, O'CONNOR, JOHNSON, KINDNESS, PLLC, 1420 FIFTH AVENUE, SUITE 2800, SEATTLE, WA, 98101-2347, US

(21) Appl. No.: 10/541,576

(22) PCT Filed: Jan. 6, 2004
(86) PCT No.: PCT/CA04/00031

§ 371 Date: Dec. 2, 2005

Related U.S. Application Data
(60) Provisional application No. 60/437,993, filed on Jan. 6, 2003.

(51) Int. Cl. A61F 2/02; A61B 17/04

(52) U.S. Cl. 600/37; 606/148

(57) ABSTRACT

The present invention provides a surgical instrument for use during a surgical procedure for the treatment of female urinary incontinence. The surgical instrument comprises a handle including a gripping mechanism (30) providing a means for the surgeon to manipulate the surgical instrument. The handle further includes a coupling means (40) enabling the interconnection of a curved needle-like element to the handle. The curved needle-like element (10) has at least two eyes therethrough, wherein one eye (80) is located in the proximity of the first end (70) of the element and a second eye (90) is located in the proximity of the opposite end (60) of the needle-like element. During the surgical procedure, surgical tape (100) is passed through one of the eyes and drawn through the body in a manner such that the surgical tape forms a suburethral sling, wherein the two ends of the surgical tape emerge from the abdominal wall.

### FIELD OF THE INVENTION

The present invention pertains to the field of surgical instruments and methods of use, in particular to a surgical instrument for the treatment of female urinary incontinence.

### BACKGROUND

Women account for approximately 11 million of incontinence cases, annually. Moreover, a majority of women with incontinence suffer from stress urinary incontinence (SUI). Women with SUI involuntarily lose urine during normal daily activities and movements, such as laughing, coughing, sneezing and regular exercise.

SUI may be caused by a functional defect of the tissue or ligaments connecting the vaginal wall with the pelvic muscles and the pubic bone. Common causes include repetitive straining of the pelvic muscles, childbirth, loss of pelvic muscle tone, and estrogen loss, wherein such a defect can result in an improperly functioning urethra. Unlike other types of incontinence, SUI is not a problem of the bladder.

Normally, the urethra, when properly supported by strong pelvic floor muscles and healthy connective tissue, maintains a tight seal to prevent involuntary loss of urine.

When a woman suffers from the most common form of SUI, however, weakened muscles and pelvic tissues are unable to adequately support the urethra in its correct position. As a result, during normal movements when pressure is exerted on the bladder from the diaphragm, for example, the urethra cannot retain its seal, permitting urine to escape. Because SUI is both embarrassing and unpredictable, many women with SUI may avoid an active lifestyle, shying away from social situations. There are many surgical procedures for the treatment of urinary stress incontinence. The suburethral sling of all of these procedures has shown the best results for all causes of urinary stress incontinence. The accepted procedures for treatment require abdominal incisions and are thus considered invasive procedures.

Tension free vaginal tape (TVT) is a relatively new surgical technique in treatment of female stress urinary incontinence and it is considered a modified suburethral sling. This procedure has revolutionized the surgical treatment of urinary stress incontinence because it is minimally invasive and has an effective cure rate of about 85-90%. The cost of disposable kits for this procedure is the main limiting factor and therefore translates into a limited number of procedures per centre, thereby limiting surgeons from offering this minimally invasive and effective procedure to every eligible patient.

U.S. Pat. No. 5,112,344 describes a method for treating female urinary incontinence without the necessity of opening the abdomen, which would require hospital care for a number of days. In this method, a tape is looped around the urethra to be implanted into the soft tissue between the vaginal wall and the abdominal wall extending above the pubis and with the ends of the tape extending into the abdominal wall. The tape is left in the body in order that fibrous scar tissue shall develop around the tape functioning as a supporting ligament in the soft tissue.

A surgical instrument for treating female urinary incontinence is also disclosed in U.S. Pat. No. 5,899,909. The instrument comprises a shank having a handle at one end thereof, and two curved needle-like elements, which are connected at one end thereof each with one end of a tape intended to be implanted into the body. These elements can be connected one at a time with the shank at the other end thereof to form a curved end portion of the shank and are intended to be passed into the body via the vagina, each element being dimensioned to extend from the inside of the vaginal wall over the back of the pubic bone to the outside of the abdominal wall. When practising the method the tape is passed into the body via the vagina first at one end and then at the other end at one side and the other, respectively, of the urethra to form a loop around the urethra, located between the urethra and the vaginal wall. The tape is extended over the pubis and through the abdominal wall and is tightened. Subsequently, the tape ends are cut at the abdominal wall, and the tape is left implanted in the body.

U.S. Pat. No. 6,406,423 discloses another method for the surgical treatment of urinary incontinence and the device for carrying out this method. The device includes a needle, an intermediate traction element, and a sleeved support tape; and the method includes forming an opening in the vaginal wall and forming suprapubic incisions, creating tracks and following one track with a needle and intermediate traction element from a suprapubic incision to the opening in the vagina, following the other track with a needle and optionally an intermediate traction element, verifying the tracks by cystoscopy, passing a sleeved tape through the tracks to form a loop under the urethra, adjusting the loop, removing the sleeve, and leaving the tape implanted.

Other surgical instruments and methods for treating female urinary incontinence are also disclosed in U.S. Pat. Nos. 6,273,852 and 6,491,703.

Each of the prior art surgical instrument devices, however, is capable of performing only a particular surgical method for treatment of female urinary incontinence. Therefore, the surgeon's selection of the surgical instrument device may be based upon the preferred surgical method. In addition, some or part of the surgical instruments of the prior art are not reusable and therefore this can increase the cost of performing the surgery. Therefore there is a need for an improved surgical instrument for the treatment of female urinary incontinence.

This background information is provided for the purpose of making known information believed by the applicant to be of possible relevance to the present invention. No admission is necessarily intended, nor should be construed, that any of the preceding information constitutes prior art against the present invention.

### SUMMARY OF THE INVENTION

An object of the present invention is to provide a surgical instrument for the treatment of female urinary incontinence and methods of use. In accordance with an aspect of the present invention, there is provided a surgical instrument for treatment of female urinary incontinence, the surgical instrument comprising: a curved needle-like element having a first and a second end, each of the first and second ends of the curved needle-like element having an eye therethrough; and a handle having a first and second end, the first end having a gripping mechanism attached thereto for enabling a person to hold and manipulate the handle, the second end of the handle having a coupling means for releaseably securing the first end of the curved needle-like element to the handle; wherein a tape to be implanted into a female body as a loop around urethra is passed through one of the eyes in the curved needle-like element and subsequently drawn into the body thereby implanting the tape within the body.

### BRIEF DESCRIPTION OF THE FIGURES

FIG. 1 is a side view of the surgical instrument according to one embodiment of the present invention.

FIG. 2 is a top view of the surgical instrument showing the coupling mechanism for the interconnection of a curved needle-like element thereto, according to one embodiment of the present invention.

FIG. 3 is a side view of the curved needle-like element according to one embodiment of the present invention.

FIG. 4 is a side view of the curved needle-like element showing the eye associated with one end thereof, according to one embodiment of the present invention.

FIG. 5 is another side view of the curved needle-like element showing the eye associated with the opposite end thereof, according to one embodiment of the present invention.

FIG. 6 is a side view of the needle-like element as illustrated in FIG. 5, wherein the element has been rotated 90 degrees.

FIG. 7 is a perspective view illustrating the commencement of an abdominal approach for a surgical technique using the surgical instrument according to one embodiment of the present invention.

FIG. 8 is a perspective view illustrating a subsequent step in the surgical procedure commenced in FIG. 7; wherein the surgical tape is passed through an eye in the curved needle-like element.

FIG. 9 is a perspective view illustrating the commencement of a vaginal approach for a surgical technique using the surgical instrument according to one embodiment of the present invention.

FIG. 10 is a perspective view illustrating a subsequent step in the surgical procedure commenced in FIG. 9, wherein the surgical tape is passed through an eye in the curved needle-like element.

FIG. 11 is a perspective view illustrating a subsequent step in the surgical procedure commenced in FIG. 9, wherein the needle-like element is removed via the abdomen.

### DETAILED DESCRIPTION OF THE INVENTION

The present invention provides a surgical instrument for use during a surgical procedure for the treatment of female urinary incontinence. The surgical instrument comprises a handle including a gripping mechanism providing a means for the surgeon to manipulate the surgical instrument. The handle further includes a coupling means enabling the interconnection of a curved needle-like element to the handle. The curved needle-like element has at least two eyes therethrough, wherein one eye is located in the proximity of the first end of the element and a second eye is located in the proximity of the opposite end of the needle-like element. During the surgical procedure, surgical tape is passed through one of the eyes and drawn through the body in a manner such that the surgical tape forms a suburethral sling, wherein the two ends of the surgical tape emerge from the abdominal wall. The suburethral sling can be positioned by using a vaginal approach or an abdominal approach, wherein the design of the surgical instrument according to the present inventions can be used for either of these surgical approaches.

Handle

The handle of the surgical instrument has a central shaft and two ends wherein one end of the handle has associated with it a gripping mechanism enabling one to hold and manipulate the surgical instrument. The opposite end of the handle has a coupling means associated therewith, which provides a means for rigidly connecting the curved needle-like element thereto. The coupling means is designed such that the curved needle-like element can be releasably connected to the handle.

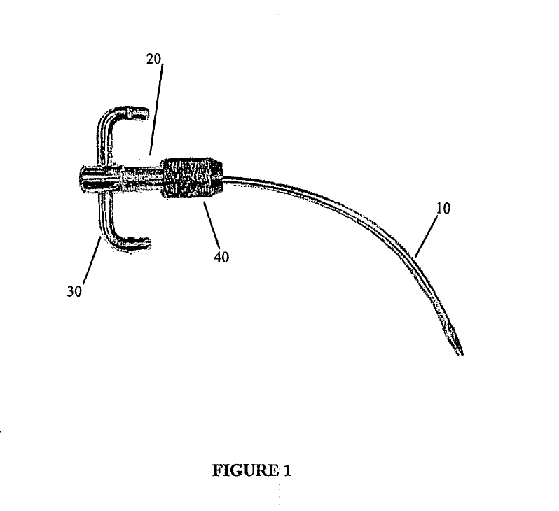

In one embodiment of the invention and with reference to FIG. 1, the gripping mechanism 30 can be in the form of two portions projecting from the handle in a common plane. These portions may project in a direction perpendicular to the longitudinal direction of the handle, however this angle of projection may vary and be dependent on an individual's preferences for a comfortable and ergonomic holding position, for example. These portions can have a further bend therein at their terminal end thereby potentially providing for a more comfortable and ergonomically effective gripping mechanism. However this bend of the terminal end of the portion is not necessarily required. The portions of the gripping mechanism may be fabricated having any number of cross sectional shapes, for example circular, elliptical, square, octagon or any other shape, wherein this cross sectional shape may change along their length. In an alternate embodiment, the gripping mechanism may be in the form of wings that project in a common plane from the handle.

The gripping mechanism can be interconnected with the shaft of the handle by welding, braising or soldering, for example. In one embodiment of the invention the gripping mechanism and the shaft of the handle can be manufactured as one unit, for example by casting the unit in a mould.

In one embodiment of the invention, the plane in which the gripping mechanism projects from the handle, is the same plane in which the curved needle-like element bends, as illustrated in FIG. 1. This common plane orientation of the gripping mechanism and the curved needle-like element may be advantageous since during use of the surgical instrument, the orientation of the surgeon's hand can be similar to that used for most surgical instruments. Therefore, this may be more comfortable and ergonomically efficient for a surgeon. The orientation of the plane of the curve of the curved needle-like element may alternately be perpendicular to that of the gripping mechanism for example, or any other orientation if so desired depending on the type of coupling means associated with the handle.

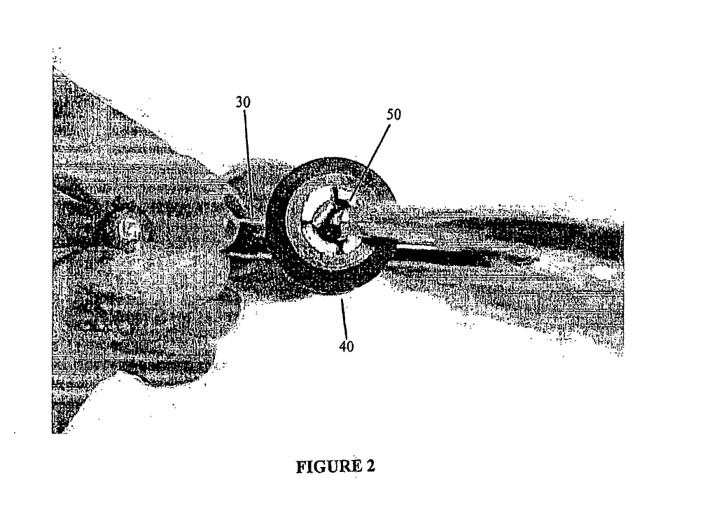

The end of the handle opposite to the gripping mechanism has a coupling means associated therewith. In one embodiment of the invention and with reference to FIG. 1, the coupling means 40 can be in the form of a chuck as is commonly used in a drill, for example. FIG. 2 illustrates another view of the coupling means along the length of the handle, wherein the jaws 50 of the coupling means associated with the handle, are visible. In one embodiment of the invention, there are two, three or four individual jaws within the coupling means. If there are three or four individual jaws associated with the coupling means, centering of the curved needle-like element within the coupling means may be automatically realised during the tightening of the coupling means towards a rigid connection between the needle-like element and the handle, for example. If, for example, there are four jaws with flat surface areas for contact a needle-like element within the coupling means and a square or rectangular cross sectional shaped needle-like element is rigidly connected to the handle, the orientation of the curve of the needle-like element may be rotated in increments of 90 degrees, for example. This feature may potentially enable a desired orientation of the curve of the needle-like element with respect to the gripping mechanism, to be realised. Alternately, if there are only two jaws within the coupling means, the needle-like element may have two planar sides in order to improve the contact between the needle-like element and the jaws of the coupling means thereby potentially ensuring the formation of a rigid interconnection therebetween. The tightening and releasing of the connection of the needle-like element to the handle is provided by a knob associated with the coupling means. The knob is rotatably connected to the handle by mating threads fabricated on the external surface area of the shaft of the handle and on the internal surface area of the knob. As would be known to a worker skilled in the art, the rotation of the knob in a first direction draws the jaws toward each other thereby tightening their grip on a needle-like element inserted between the jaws, by a wedging action for example. The rotation of the knob in the opposite direction will release the needle-like element. The external surface area of the knob can be fabricated with a friction enhancing relief pattern, for example grooves or a mesh pattern, for improved grip of the knob during use.

In one embodiment of the invention, biasing means are associated with the jaws of the coupling means forcing the jaws into an open orientation thereby providing for ease of insertion and removal of the needle-like element into and out of the coupling means of the handle. The biasing means can be a spring fabricated from surgical steel or stainless steel.

All of the parts of the handle as described so far are intended to be used several times and therefore should consist of a material which can be sterilised, for example by autoclaving or flash sterilisation. For example, the handle can be manufactured from stainless steel or other material, which has the specified properties as, would be readily understood by a worker skilled in the art of surgical instrument manufacture.

In one embodiment of the present invention, the handle can be readily disassembled, for example removing the jaws from the coupling means, in order that the sterilisation process may be performed in a potentially more complete manner.

Curved Needle-Like Element

The curved needle-like element has a first and second end wherein in the proximity of both the first and second ends, an eye has been provided therethrougli Since eyes are provided at both ends of the curved needle-like element, a surgical technique for the treatment of female incontinence involving either the vaginal approach or the abdominal approach to the surgery may be performed using the surgical instrument of the present invention.

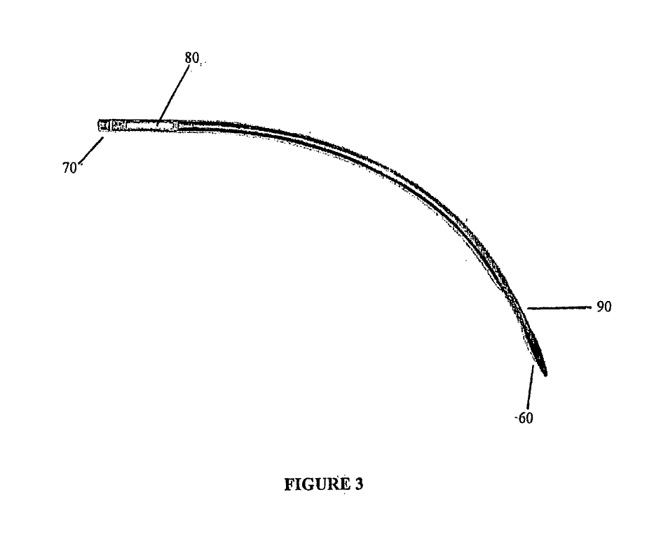

With reference to FIG. 3, one embodiment of the curved needle-like element is illustrated. The needle-like element 10 has a first end 70 that comprises a straight portion and the element extends therefrom over substantially a quarter of a circle to the other, free end 60 thereof in order to follow substantially the profile of the pubis between the vagina and the abdominal wall, for example. The needle-like element can have a circular cross section and a smooth surface area that may also be polished. Alternately, the needle-like element may have an elliptical cross sectional shape or optionally a square, rectangular or octagonal cross section with rounded edges, for example. The needle-like element can taper towards the free end 60 thereof where the needle forms a point that may be conical in shape or may optionally be faceted. At both the first end 70 and the free end 60, eyes 80 and 90, respectively, have been formed within the cross section of the needle-like element. The first end 70 having the straight portion is inserted into the coupling means associated with the handle enabling the rigid connection thereto, upon the rotation of the knob.

The shape of the eyes provided within the needle-like element can take any cross sectional shape for example a circle, ellipse, slot or elongated slot. In one embodiment of the invention, the cross sectional shape of the eyes is an elongated slot thereby potentially making the passing of a surgical tape therethrough easier.

The eyes, which are formed within the needle-like element at the first end and second end (free end) thereof, can be oriented in any direction with respect to the cross section of the needle-like element. The orientation of the eye within the needle-like element may be determined by the manufacturing technique of the element itself.

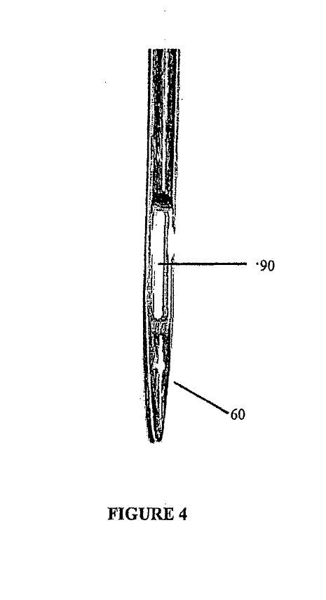

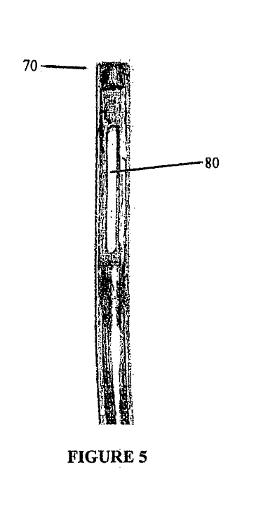

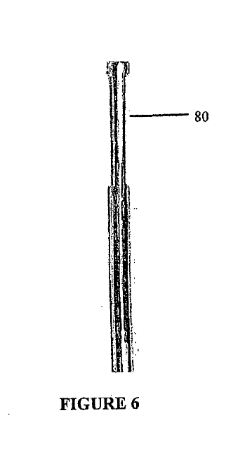

FIG. 4, illustrates the free end of the needle-like element showing the eye 90 associated therewith, according to one embodiment of the invention. FIG. 5, illustrates the first end of the needle-like element showing the eye 80 associated therewith according to one embodiment. FIG. 6 illustrates the first end of the needle-like element rotated 90 degrees with respect to FIG. 5, based on one embodiment of the present invention. The first end of the needle-like element may have two flat edges surrounding the eye formed therein and protrusions at the tip of the first end, wherein the flat edges and the protrusions may provide for a more secure connection between the coupling means associated with the handle and the first end of the needle-like element. Optionally, the cross sectional shape of the first end of the needle-like element may not change along its length, except at the second end (free end) of the needle-like element wherein the cross section can taper.

The curved needle-like element as described so far is intended to be used several times and therefore should consist of a material which can be sterilised, for example by autoclaving or flash sterilisation. For example, the curved needle-like element can be manufactured from stainless steel or other material that has the specified properties as would be readily understood by a worker skilled in the art of surgical instrument manufacture.

Upon the interconnection of the needle-like element and the handle, they form a rigid unit that can be controlled with great precision when the surgical instrument is used for the surgery applying the methods of the present invention.

Surgical Tape

The surgical tape of the present invention is implanted into the body in order to form a suburethral sling that may enable the treatment of female urinary incontinence. The surgical tape used to form the sling can be an autologous material such as rectus fascia or a heterologous material, which may be from an animate source or may be a synthetic material.

In one embodiment of the present invention, the tape comprises a mesh or netting forming openings of the order of 1 mm in order that fibroblasts can be able to grow into the tape for anchoring of the tape in surrounding tissue. A suitable material for the tape is PROLENE™, a knitted polypropylene mesh having a thickness of 0.7 mm manufactured by Ethicon, Inc., Sommerville, N.J., USA. This material is approved by FDA in USA for implantation into the human body.

In another embodiment of the present invention, the tape can be knitted or woven more closely than the tape mentioned above and can be of such material that the tape after a shorter or longer period of time may be completely resorbed by the body. By the development of fibroblast proliferation stimulated by the tape, the reinforcement of the tissue required in order to restore the urinary continence may be obtained.

In one embodiment, the material of the tape can be coated with a fibroblast stimulating substance, e.g. an enamel matrix derivative.

Method of Use by the Abdominal Approach

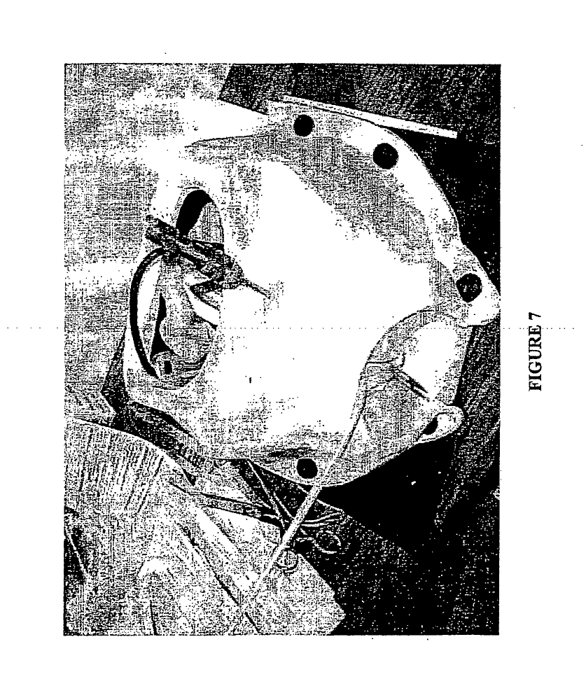

Initially the curved needle-like element and the handle are rigidly connected together. The first step of the surgery for implanting the tape is disclosed in FIG. 7 and comprises the penetration of the abdominal wall by the needle-like element, an incision having first been made in the abdominal wall on one side of the pubic bone. The needle-like element is guided through the soft tissue at one side of the urethra passing behind the pubic bone and subsequently through the vaginal wall, via an incision therein.

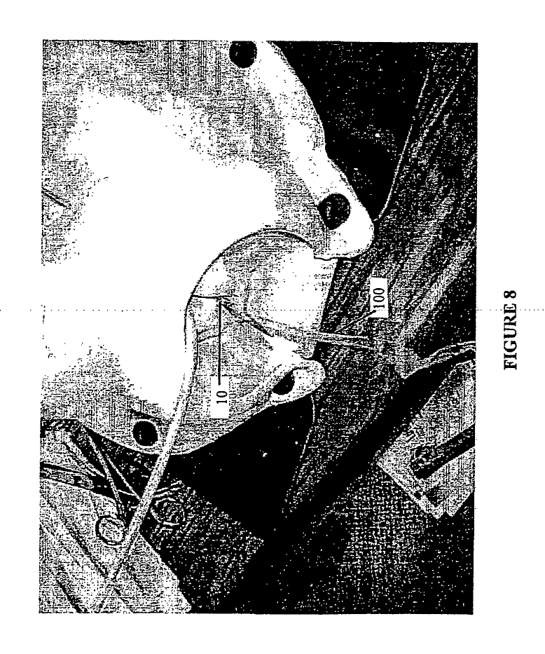

A first end of the surgical tape 109 is inserted into the eye of the needle-like element located at the free end thereof as shown in FIG. 8. The needle-like element is subsequently withdrawn from the abdominal wall together with the first end of the surgical tape. The withdrawal of the first needle-like element may occur after the insertion of a second needle-like element as discussed below, for example.

A second needle-like element, or the original element, potentially having been resterilised, is rigidly connected to the handle of the surgical instrument. Similar as previously performed, the needle is passed through a second incision in the abdominal wall on the opposite side of the pubic bone, passing on the opposite side of the urethra and passing out the incision in the vaginal wall. The second end of the surgical tape is inserted into the eye and the needle-like element located at the free end. The needle-like element is subsequently withdrawn from the second abdominal incision together with the second end of the surgical tape.

In one embodiment, prior to the withdrawing of a needle-like element, the path of the needle-like element can be examined using cystoscopy in order that it can be verified that there has not been any puncture or damage to either the urethra or the bladder. This check can be performed once if two needle-like elements are used for the surgery, however two checks may be performed if only one needle-like element is used to perform the procedure. If two needle-like elements are used, the withdrawal of both needle-like elements and the drawing of the surgical tape into the body will occur upon inspection of the urethra and the bladder using cystoscopy.

The surgical tape is now located on either side of the urethra forming a loop there around. This loop can be adjusted thereby potentially optimising the support and/or restriction of the urethra. The excess of the tape on the outside of the abdominal wall can be cut off and the tape may be left as an implant in the body to form an artificial ligament, for example and potentially providing the support for the urethra as required to restore urinary continence.

Method of Use by the Vaginal Approach

Initially the curved needle-like element and the handle are rigidly connected together.

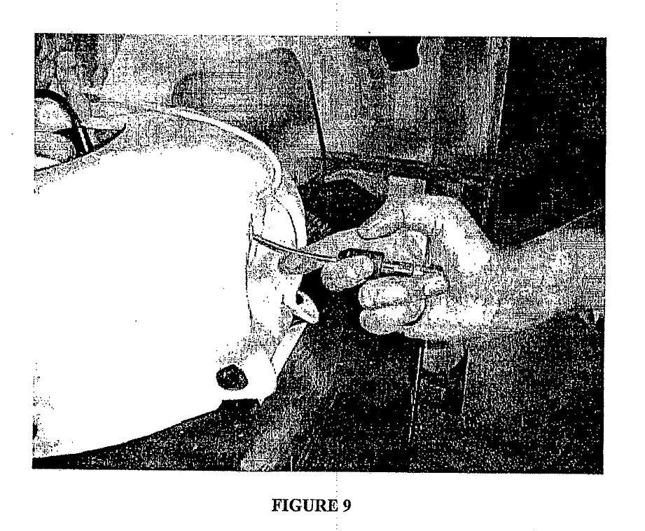

The first step of the surgery for implanting the tape is disclosed in FIG. 9 and comprises the penetration of the vaginal wall by the needle-like element, an incision having first been made in the vaginal wall. The needle-like element is guided through the soft tissue at one side of the urethra passing behind the pubic bone and subsequently through the abdominal wall above the pubic bone, via an incision within the abdominal wall.

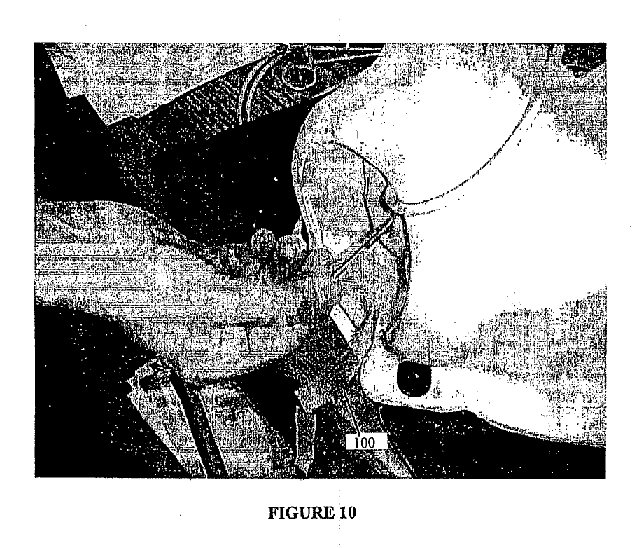

The handle of the surgical instrument is released from the needle-like element exposing the eye at the first end of the element. A first end of the surgical tape 100 is inserted into this eye of the needle-like element as shown in FIG. 10. The needle-like element is subsequently withdrawn from the abdominal wall by means of the surgeon's grasp or forceps. The first end of the surgical tape, which was inserted into the eye of the needle-like element, is also pulled through the abdominal wall. The withdrawal of the first needle-like element may occur after the insertion of a second needle-like element as discussed below, for example.

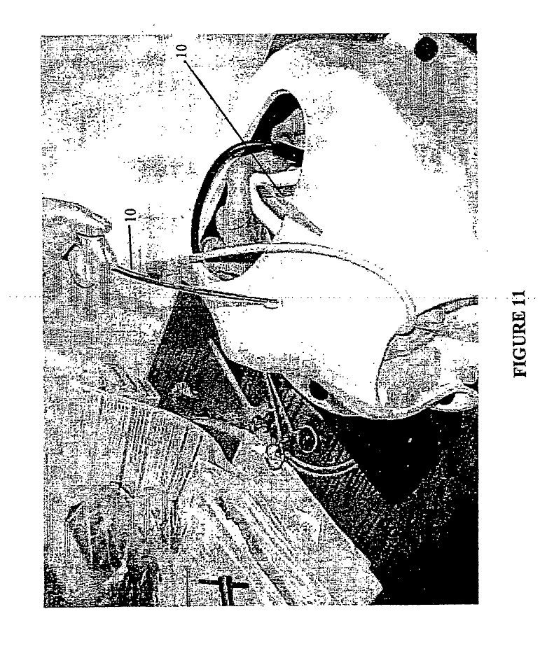

A second needle-like element, or the original element, potentially having been resterilised, is rigidly connected to the handle of the surgical instrument. As initially performed, the needle is passed through the incision in the vaginal wall passing on the opposite side of the urethra and passing out an incision on the opposite side of the pubic bone, as illustrated in FIG. 11. The handle of the surgical instrument is released from the needle-like element exposing the eye at the first end of the element. The second end of the surgical tape is inserted into this eye and the needle-like element is withdrawn from the second abdominal incision together with the second end of the surgical tape.

In one embodiment, prior to the withdrawing of a needle-like element, the path of the needle-like element can be examined using cystoscopy in order that it can be verified that there has not been any puncture or damage to either the urethra or the bladder. This check can be performed once if two needle-like elements are used for the surgery, however two checks may be performed if only one needle-like element is used to perform the procedure. If two needle-like elements are used, the withdrawal of both needle-like elements and the drawing of the surgical tape into the body will occur upon inspection of the urethra and the bladder using cystoscopy.

The surgical tape is now located on either side of the urethra forming a loop there around. This loop can be adjusted thereby potentially optimising the support and/or restriction of the urethra. The excess of the tape on the outside of the abdominal wall can be cut off and the tape may be left as an implant in the body to form an artificial ligament and potentially providing the support for the urethra as required to restore urinary continence.

### EXAMPLE

Randomized Control Trial Comparing Modified TVT Using Reusable Pubovaginal Sling Device in Treatment of Urinary Stress Incontinence

A randomized control trial was conducted comparing the surgical procedures, using the reusable pubovaginal sling device of the present invention (modified TVT procedure) and a standard Gynecare device (standard Gynecare TVT procedure), for the treatment of SUI.

Fifty patients with SUI were randomized into two groups for surgical treatment of SUL Each patient had a full history, gynecological and pelvic examination carried out. Each patient underwent a full urodynamic assessment including uroflometery and multichannel subtracted cystometry. The diagnosis of stress urinary incontinence was confirmed with objective demonstration of incontinence and leak point pressure analysis.

The first group of 25 patients underwent the modified TVT procedure using the reusable pubovaginal sling according to the present invention. The second group of 25 patients underwent the Gynecare TVT procedure using a standard Gynecare TVT device.

TABLE 1
Patient Demographics

| Variable | Gynecare TVT | Modified TVT | P-value |
|---|---|---|---|
| Number of Patients | 25 | 25 | |
| Mean Age (yrs.) | 51.08 | 51.88 | 0.78 NS |
| Parity | 2.5 | 2.7 | 0.74 NS |
| Mean BMI | 28.0 | 29.80 | 0.31 NS |
| Incontinence Type: | | | 0.77 NS |
| (Number of Patients) | | | |
| Stress | 16 | 15 | |
| Mixed | 4 | 5 | |
| Recurrent | 5 | 5 | |
| Mean Leak Point | 74.16 | 71.25 | 0.64 NS |
| Pressure | | | |

The two groups were comparable in patient demographics. The mean age for the Gynecare TVT group was 51.08 years compared to 51.88 years for the modified TVT group. This was not significant with a P-value of 0.78 (not significant). The parity similarly was 2.5 in Gynecare TVT group compared to 2.7 in the modified TVT Gynecare with a P-value of 0.74 (not significant). The body mass index in the Gynecare TVT group was 28.0 compared to 29.80 in the modified TVT group and a P-value of 0.31 (not significant). The type of incontinence in the patients, such as stress, mixed and recurrent, were comparable between the two groups with numbers of 16, 4 and 5 patients in the Gynecare. TVT group and 15, 5 and 5 patients in the modified TVT group, respectively (P-value 0.77 (not significant)). The leak point pressure in each group was 74.16 in the Gynecare TVT group compared to 71.25 in the modified TVT group with a P-value of 0.64 (not significant) (See Table 1).

TABLE 2
Surgery times

| | Gynecare TVT | Modified TVT | P-value |
|---|---|---|---|
| Operation Time | 27.76 | 25.62 | 0.42 NS |
| (Minutes) | | | |
| Anaesthesia Time | 20.26 | 23.60 | 0.12 NS |
| (Minutes) | | | |
| Void Time | 0.60 | 0.45 | 0.54 NS |
| (Days) | | | |
| Length of Stay | 0.84 | 0.72 | 0.70 NS |
| (Days) | | | |

The surgery time was comparable in the two groups with 27.76 minutes in the Gynecare TVT group compared to 25.62 minutes in modified TVT group and a P-value of 0.42 (not significant). The anaesthesia time, similarly, was comparable with 20.26 minutes in the Gynecare TVT group comparable to 23.60 minutes in the modified TVT group and a P-value of 0.12 (not significant). The resumption to normal voiding was comparable in the two groups with 0.60 days in the Gynecare TVT group compared to 0.45 days in modified TVT group and a P-value of 0.54 (not significant). The length of hospital stay was similar in the two groups with 0.84 days in the Gynecare TVT group compared to 0.72 days in modified TVT group with a P-value of 0.70 (not significant) (See Table 2).

TABLE 3
Equipment costs and success rate.

| | Gynecare TVT | Modified TVT | P-value |
|---|---|---|---|
| Equipment Cost | 675 | 10 | <0.0001 |
| ($CDN) | | | |
| Total Cost | 1,712 | 997 | 0.01 |
| ($CDN) | | | |
| Surgical Cure | 21 (84%) | 22 (88%) | 1.00 |
| 6-weeks post-op. | | | |
| Surgical Cure | 22 (88%) | 22 (88%) | NS |
| 1-year post-op. | | | |

The equipment cost of the Gynecare TVT group included the cost of the disposable surgical kit for the procedure. The cost of this kit was six hundred and seventy-five Canadian dollars ($675) compared to only ten dollars ($10) for a piece of mesh in modified TVT group. This difference was very significant with a P-value of less than 0.0001. The total cost including cost of surgery, anaesthetist fee, equipment cost and hospital stay was approximately 50% less in the modified TVT group. This value was with a mean of one thousand seven hundred and twelve dollars ($1,712) per patient in Gynecare TVT group compared to only nine hundred and ninety-seven dollars ($997) in modified TVT group. This difference again was statistically significant with a P-value of 0.01. The procedures were comparable as far as success was concerned at six weeks postoperatively and one year postoperatively. The six weeks success included 21 patients cured out of 25 (84%) in the Gynecare TVT group compared to 22 patients cured out of 25 (88%) in the modified TVT group. This was not significant with a P-value of 1.00. The surgical cure for one-year postoperatively was the same for both groups with 22 patients cured in each group and a success rate of 88% (see Table 3).

The modified pubovaginal TVT sling of the present invention can be as effective as the standard Gynecare TVT procedure. The procedure of modified TVT using a reusable pubovaginal sling device however has no upfront cost, apart from the initial purchase price of the device itself and the total cost is 50% less compared to standard TVT procedure.

The embodiments of the invention being thus described, it will be obvious that the same may be varied in many ways. Such variations are not to be regarded as a departure from the spirit and scope of the invention, and all such modifications as would be obvious to one skilled in the art are intended to be included within the scope of the following claims.

I claim:

1. A surgical instrument for treatment of female urinary incontinence, the surgical instrument comprising:
a) a curved needle-like element having a first and a second end, each of the first and second ends of the curved needle like element having an eye therethrough; and
b) a handle having a first and second end, the first end having a gripping mechanism attached thereto for enabling a person to hold and manipulate the handle, the second end of the handle having a coupling means for releaseably securing the first end of the curved needle-like element to the handle;
wherein a tape to be implanted into a female body as a loop around urethra is passed through one of the eyes in the curved needle like element and subsequently drawn into the body thereby implanting the tape within the body.

2. The surgical instrument according to claim 1, wherein the curved needle-like element is manufactured from a material resistant to standard sterilisation techniques.

3. The surgical instrument according to claim 1, wherein the first end of the needle-like element comprises two opposite planar surfaces surrounding the eye at the first end.

4. The surgical instrument according to claim 1, wherein the first end of the needle-like element further comprises protrusions providing a means for secure connection between the needle-like element and the coupling means.

5. The surgical instrument according to claim 1, wherein the eyes at the first and second end have a cross section shape selected from the group comprising circle, ellipse, slot and elongated slot.

6. The surgical instrument according to claim 1, wherein the gripping mechanism comprises two portions projecting in a common plane from opposite sides of the handle.

7. The surgical instrument according to claim 1, wherein the curved needle-like element bends in a plane parallel to the common plane.

8. The surgical instrument according to claim 1, wherein the curved needle-like element bends in a plane perpendicular to the common plane.

9. The surgical instrument according to claim 1, wherein the coupling means is a chuck.

10. The surgical instrument according to claim 1, where the coupling means comprises at least two jaws for frictional interconnection with the curved needle-like element.

11. The surgical instrument according to claim 6, wherein the coupling means further comprises a biasing means thereby providing ease of insertion and removal of the curved needle-like element from the coupling means.

12. Use of the surgical instrument according to claim 1 for treatment of female urinary incontinence.
